(19) United States
(12) Patent Application Publication
(10) Pub. No.: US 2017/0157293 A1
(43) Pub. Date: Jun. 8, 2017

(54) COMPOSITE PART FOR ENDOSSEOUS IMPLANTATION AND METHOD FOR MANUFACTURING SAME

(71) Applicant: JEAN-PIERRE COUGOULIC, PORNICHET (FR)

(72) Inventor: JEAN-PIERRE COUGOULIC, PORNICHET (FR)

(73) Assignee: Catherine Cadorel, Pornichet (FR)

(21) Appl. No.: 14/232,579

(22) PCT Filed: Jul. 13, 2011
(86) PCT No.: PCT/EP2011/062011

§ 371 Date: Apr. 28, 2014

(51) Int. Cl. A61L 27/44; A61L 27/48; A61L 27/58; A61L 27/54; A61L 27/46; A61K 6/033; A61K 6/027; A61K 6/087; A61K 6/00; B29B 7/00; B29B 7/82; B29B 7/90; B29B 9/06; B29B 9/14; B29C 45/00; B29C 71/00; A61F 2/30; A61L 27/18

(52) U.S. Cl. CPC A61L 27/446; A61L 27/18; A61L 27/48; A61L 27/58; A61L 27/54; A61L 27/46; A61K 6/033; A61K 6/027; A61K 6/087; A61K 6/0067; B29B 7/002; B29B 7/82; B29B 7/90; B29B 9/06; B29B 9/14; B29C 45/0053; B29C 45/0001; B29C 45/0005; B29C 71/0009; A61F 2/30965; A61L 2430/02; A61L 2400/12; A61L 2300/10; A61L 2300/112; B29K 2071/00

(57) ABSTRACT

A part adapted for in vivo endosseous implantation made up of a material comprising a thermoplastic organic binder and a fiber charge. The fibers located in a surface layer of the part are mostly delaminated from the binder over all or part of their length. Also, a method for manufacturing such a part.

The invention relates to a part designed to be implanted in bone tissue such as a dental implant, a prosthesis or a bone filling for medical or veterinary purposes, wherein said part is made up of a material which, combined with a particular manufacturing process, speeds up its osseointegration into the receiving tissue.

Different implants made up of biocompatible polymer are known in the prior art, where the making process allows the creation of a surface texture made up of micropores that are conducive to cell colonization by the receiving tissue, thus speeding up the osseointegration of said implant.

These implants of the prior art provide very satisfactory results; however, the thickness of the osseointegration layer obtained by that mechanism, which corresponds to the depth of the surface micropores, is about 1000 nanometers (1 μm). However it is generally accepted that a larger thickness of interpenetration of the tissue and implant, at least ranging from 1 μm to 10 μm, is preferable, espacially when the elasticity characteristics of the implant and those of the receiving tissue are different. Such increased interpenetration is thus particularly sought when the implant is reinforced, particularly by fibers and more particularly at the beginning of the osseointegration process when the cortical bone in formation does not yet have an elasticity modulus comparable to that of the implant.

Besides, such microporous surface textures are difficult or even impossible to make using cost-effective implant manufacturing processes such as injection molding.

The invention is aimed at remedying these drawbacks of the prior art by proposing an implant and a cost-effective process for making the implant in such a way so as to increase the interpenetration depth between the implant and the receiving bone tissue.

To that end, the invention discloses a part adapted to in vivo endosseous implantation made up of a material comprising:

a thermoplastic organic binder; and

a fiber charge,

wherein the fibers are mostly delaminated from the binder over part of their length in a surface layer of said part.

Fibers means microfibers, nanofibers or nanotubes with a length to thickness ratio greater than 10. Microfibres are fibers with thickness of about a micrometer or a micron, that is to say the thickness substantially ranges between 10⁻⁶ and 10⁻⁵ meters. Nanofibres and nanotubes are fibers with thickness of about a nanometer, that is to say substantially ranging between 10⁻⁶ and 10⁻⁸ meters.

The delamination of fibers in the surface layer makes it possible to create interstices that act as conduits and by capillarity in the thickness of that layer to carry organic fluids into it, thus speeding up cell colonization of the layer. The nature of the fibers also makes it possible to favor and speed up, by absorption, the transport of such organic fluids.

The invention can be implemented according to the advantageous embodiments described below, which may be considered individually or in any technically operative combination.

Advantageously, the thermoplastic binder is made of polyetheretherketone (PEEK), the biocompatibility properties of which are known.

Also advantageously, the fiber charge comprises fibers made of a polymer of the family of aromatic polyamides, which also have excellent biocompatibility properties combined with high mechanical properties. More particularly, poly(amide-imide) fibers with a glass transition temperature close to the injection molding temperature of PEEK allow, due to their ease of deformation during injection, a homogeneous distribution of the fibers in the implant even when they are relatively long.

The effect of conduction of provided by the delaminated fibers in the surface layer makes it possible to obtain a thickness of said layer of at least 2 μm, that is to say it is significantly greater than what can be obtained with implants made by plastic injection comprising surface micropores without a delamination effect.

Advantageously, the material that makes up the implant comprises, in addition to fibers, a charge of components made from calcium and phosphorous. These resorptive compounds favor osseointegration and healing.

Advantageously, the charge in calcium-based component is made up of tricalcium phosphate Ca₃(PO₄)₂ with a hexagonal β structure. These tricalcium phosphate compounds are transformed during the injection molding operation into resorptive nonstoichiometric calcium apatite crystals.

Advantageously, the material making up the implantable part may also contain a zeolite charge. These zeolites do promote electrostatic links with the implantation environment and ionic bonding with that environment. Such a charge further helps make the material radio-opaque.

Advantageously, the fiber charge comprises fibers made of calcium silicates (Ca₂SiO₄). The presence of these fibers at the surface of the part speeds up the absorption of interstitial fluids in the implantation environment and therefore the cell colonization of the surface of the part.

The invention also relates to a method for manufacturing such an implant, wherein said method comprises the steps of:
a) mixing a thermoplastic polymer and a fiber charge by extrusion and granulation;
b) molding the part by injection in a mold comprising a cavity with an appropriate shape from the granulate obtained in step (a);
c) submitting the blank obtained in step (b) to ultrasonic pickling baths for a time appropriate for delaminating the fibers in a surface layer.

The plastic injection method makes it possible to cost-effectively produce this type of implant with finished dimensions at the end of the molding operation, in large quantities. It further makes it possible to direct the fibers by the flow of material penetrating into the mold and thus obtain an optimal reinforcement effect, even when the shapes of the implants are complex. The physical-chemical treatment combining the chemical effect of the baths and the mechanical effect of the ultrasound makes it possible to simultaneously pickle/etch the surface of the implant in order to eliminate any pollution relating to the injection molding method and to produce, in the surface layer, the fiber delamination capable of producing the desired conduction effect.

In order to obtain a part that comprises, in addition to fibers, compounds made from calcium and phosphates, the invention also relates to a method for making a granulate or compound comprising the steps of:
making a first granulate or compound by mixing the polymer binder with compounds introduced in the form of powders by extrusion and granulation; and
mixing that first granulate with fibers during a second extrusion and granulation operation so as to form the granulate used for the injection molding operation.

Advantageously, the zeolite charge can also be introduced during the making of the first granulate.

The fiber charge advantageously ranges between 5% and 15% by mass of the mixture. That proportion results in a significant reinforcement of the final part, at the same time allowing its manufacture using the injection method, and allowing the mixing of the fibers with the polymer binder by extrusion and granulation or compounding, whether or not the binder has first been charged with compounds containing calcium and/or zeolites.

The invention also relates to a granulate or compound for manufacturing a fiber-reinforced implant by plastic injection, which granulate comprises:
a polyetheretherketone (PEEK) polymer binder;
a 10% to 20% charge by mass of compounds containing calcium and zeolites;
a 5% to 15% fiber charge.

This type of granulate can be used directly for plastic injection manufacturing according to step (b) of the method according to the invention.

In a first embodiment of the granulate according to the invention, the fiber charge comprises fibers made up of a poly(amide-imide), the glass transition temperature of which is equal to or below the injection temperature of PEEK.

In a second embodiment of the granulate according to the invention, the fiber charge comprises fibers made of calcium silicate (Ca₂SiO₄).

After molding, the part is pickled in a succession of ultrasonic baths in order to make it suitable for in vivo implantation and to advantageously create a surface layer on it that favors osseointegration in the receiving environment.

Advantageously, pickling is carried out in a succession of baths comprising, in the stated order:
immersion in a bath subjected to ultrasound adapted to reduce particles containing iron;
immersion in a solvent of the binder subjected to ultrasound, which is inert in respect of the fibers.

The first bath makes it possible to eliminate surface pollution by metal particles from the injection press and mold. By only dissolving the binder, the second bath makes it possible, with the combined action of the ultrasound, to create separations or delamination between the fibers and the matrix in a surface layer. The order of the baths is important, in that the acid can also have a reducing effect on the fibers and/or the charge of compounds containing calcium or zeolites present on the surface. By first attacking with acid, the subsequent action of the solvent makes these compounds appear once again on the surface.

According to an advantageous embodiment, more particularly suitable for the embodiment in which the material making up the implant comprises fibers and a charge of zeolites and compounds containing calcium in a PEEK matrix, the pickling operation comprises immersion in the following baths:
Hydrochloric acid
Acetone
Hydrogen peroxide

Separated by rinsing in a bath of water that is also subjected to ultrasound. The last hydrogen peroxide bath particularly makes it possible, when the implantable part according to the invention contains calcium silicate fibers, to create a layer of silica (SiO2) on the surface of those fibers emerging at the surface of the part. By absorbing moisture, that silica layer favors the conduction of organic fluids in the surface layer of the implant.

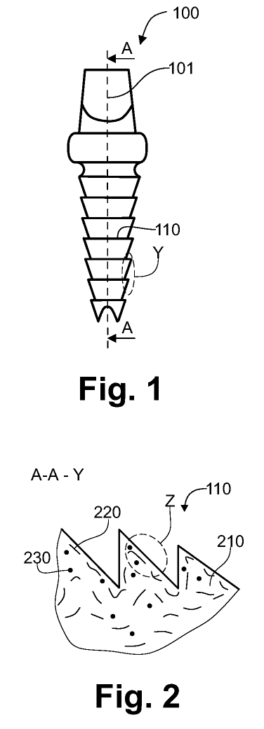

The invention will now be described in greater detail in the context of preferred embodiments, which are in no way limiting, shown in FIGS. 1 to 4, wherein:

FIG. 1 is a front view of an endosseous dental implant according to an exemplary embodiment of the invention;

FIG. 2 shows a detail Y defined in FIG. 1 along a section AA also defined in FIG. 1;

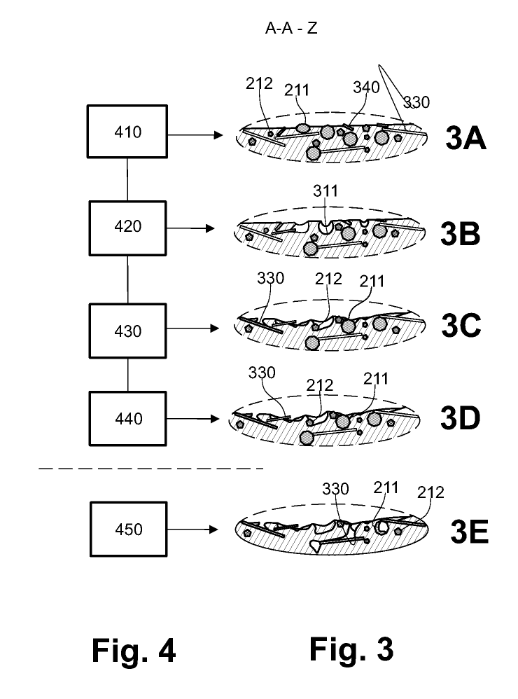

FIG. 3 represents a detail Z defined in FIG. 2 along a section AA of the surface of an implant according to an exemplary embodiment of the invention during the phases of making and implanting said implant in the bone, in FIGS. 3A to 3E;

and FIG. 4 is a chart of the different phases of making and implementing an implant according to the invention.

In FIG. 1, an example of implant (100) with a complex shape can be made cost-effectively using a plastic injection molding method. That exemplary embodiment, which is in no way limiting, represents an application of the invention to the making of a dental implant. Said dental implant comprises an upper part (101) designed to receive superstructures such as a core build-up and a so-called lower part (110) designed to be implanted in bone tissue. The lower part (110) may optionally comprise relief such as ridges adapted to favor its primary mechanical bonding in a location such as a bore made in the receiving bone tissue. The size of such primary bonding ridges or relief features is approximately a millimeter. Said implant is mostly made of thermoplastic polymer with high biocompatibility properties and is suitable for implementation using injection molding techniques. As a non-limiting example, said polymer may be made of polyetheretherketone or PEEK as distributed commercially by VICTREX® under the name VICTREX® PEEK 150G®. Advantageously, the binder may be made of material simultaneously comprising PEEK, charges of compounds containing calcium and zeolites such as the material described in the French patent

In FIG. 2, according to a first detailed sectional view, the material making up the implant comprises a matrix (210) or binder in PEEK, particles (230) of compounds containing calcium with a diameter of about 1 μm (10⁻⁶ meter) and reinforcing fibers (220). In this exemplary embodiment, the reinforcing fibers (220) are made of poly(amide-imide), such as fibers available commercially under the name KERMEL® TECH from KERMEL®, 20 rue Ampere, 68027 Colmar, France. In one exemplary embodiment using microfibers, these have a diameter of approximately 7 μm with a length of approximately 700 μm (0.7 mm). Because the implant is obtained using a plastic injection method, the injection temperature of the PEEK is equal to or greater than the glass transition temperature of that polymer so that the fibers are easily deformable at the injection temperature and that they follow substantially the flow of material.

The fiber charge may, in an advantageous embodiment, additionally or exclusively contain calcium silicate fibers (Ca₂SiO₄) (not shown in FIG. 2). The material is rigid at the injection temperature and is thus not deformed at that temperature. Therefore, the calcium silicate fibers are preferably smaller in size, with a diameter of about 1 μm and a length of about 10 μm to 50 μm. In order to prevent jamming during the injection process, the total fraction of fibers, including all fibers, must not exceed 15% by mass.

Advantageously, the charge of compounds (230) comprising calcium is made of tricalcium phosphate Ca₃(PO₄)₂ in β phase. The β phase of tricalcium phosphate is the crystalline phase with a hexagonal structure that is stable at a low temperature.

By combining with the moisture contained in the tricalcium phosphate powder, PEEK and possibly zeolites, the compound undergoes a transformation during the injection molding operation according to the following reaction:

4Ca₃(PO₄)₂+4(H₂O)=>3((Ca₃(PO₄)₂)(OH)₂Ca+2HPO₄+½O₂

3((Ca₃(PO₄)₂)OH₂)Ca is hydroxyapatite. This apatite is completely nonstoichiometric, and thus resorptive, giving the material of the implantable part according to the invention integration properties, similar to a transplant, in bone tissue.

To that end, the powders used during injection are not dehydrated. They can advantageously be rehydrated, or orthophosphoric acid (H₃PO₄) may be added to them to favor that reaction.

In FIG. 3, the observation of the surface at an ever smaller scale makes it possible to analyze the morphology of the surface depending on the implementation steps of the method and the implantable part in the invention, with the steps of the method stated in FIG. 4.

In one exemplary embodiment, the implant is obtained by a first step aimed at obtaining a granulate mixing:

80% by weight of PEEK

10% by weight of tricalcium phosphate (Ca₃PO₄)

10% by weight of titanium dioxide (TiO₂)

All the components are mixed by extrusion at a temperature ranging between 340° C. and 400° C.

By granulation of the extrusion, a first granulate is obtained, which is mixed with 10% by mass of poly(amide-imide) fibers of the KERMEL® TECH type and calcium silicate fibers according to the same extrusion and granulation method.

The second granulate obtained in this manner is used for plastic injection molding (410) of the implant. Molding takes place at a temperature ranging between 340° C. and 400° C. at a pressure ranging between 70 and 140 MPa, wherein the mold is heated to a temperature above the glass transition temperature of PEEK or a mold pre-heating temperature of approximately 160° C.

The vitreous transition temperature of fibers of the KERMEL® type is 340° C., and they are thus deformable at the injection temperature, which enables them to follow the flow of material and be distributed evenly in the granulate during the extrusion and granulation operation and in the part during the injection molding operation.

At the end of the molding operation (410) in FIG. 3A, the surface of the implant is substantially smooth and comprises some particles (211) of compounds comprising calcium and zeolites (212) emerging slightly. Fibers (330), calcium silicate in this case, are also present in the vicinity of the surface and possibly emerge slightly from said surface. The surface of the implant also comprises metallic inclusions (340) from contact with the mold and the screw of the injection press.

At the end of the molding operation, the implant is subjected to a series of chemical etching/pickling baths subjected to ultrasound. For example, the following protocol provides good practical results, with the application of ultrasound at a frequency of 42 kHz:

HCl 30%: 35 minutes

H₂O: 10 minutes (or rinsing)

C₃H₆O (acetone): 35 minutes at the boiling temperature of acetone

Drying of the implant by acetone evaporation

H₂O₂ 30%: 35 minutes

NaClO: 35 minutes

H₂O: 10 minutes (or rinsing)

The implant is then immersed, also under ultrasound, in sterilizing agents:

GIGASEPT® 12%: 35 minutes

H₂O ppi: 35 minutes

Immersion in the GIGASEPT® solution is optional.

In a first step (420) the implant is subjected to acid etching in hydrochloric acid. Such etching/pickling is chiefly aimed at removing the metallic inclusions. After that etching/pickling operation, the surface of the implant in FIG. 3B is free of metallic inclusions, and also the particles containing calcium that were emergent, leaving corresponding cavities (311) in their place.

After rinsing, the next step (430) consists in immersing the implant in an acetone bath, also subjected to ultrasound. In FIG. 3C, at the end of that step (430) a thickness of PEEK is dissolved, making initially underlying particles (211, 212) of compounds including calcium and zeolites visible. The ultrasound also tends to delaminate the fibers (330) emerging at the surface from their implantation in the matrix.

After rinsing, the next step (440) consists in immersing the implant in a hydrogen peroxide bath, also subjected to ultrasound. That bath does not fundamentally modify the morphology of the surface, in FIG. 3D. On the other hand, it has an effect on the surface of the calcium silicate fibers where it tends to form silica (SiO₂) by oxidation at their surface.

Advantageously, the implant is then inserted in a sterilization sleeve for autoclave treatment. It then undergoes a sterilization cycle at a temperature of about 135° C. for 10 minutes, under pressure of about 2150 hPa. That autoclave sterilization operation contributes to the surface pickling function; it may be associated with ethylene oxide or gamma ray treatment. Further, it favors the crystallization of particles of calcium compounds on the surface. At the end of sterilization, the implant is packaged in sterile packaging and is ready to be implanted in bone tissue.

During the implantation (450) of said implant in the tissue, organic fluids such as blood will follow by capillarity the delamination between the fibers and the matrix, whether the fibers are KERMEL fibers or calcium silicate fibers, FIG. 3E. In the case of calcium silicate fibers, the silica present on the surface of these fibers absorbs the fluids and thus favors conduction under the surface. On the surface and by conduction by the fibers, under that surface the calcium-based compounds (211) come in contact with these organic fluids. The resorptive nature of these compounds thus favors cell colonization leading to the grafting of the surface of the implant in the bone tissue.

The application of the surface treatment to an implant of the prior art that only contains calcium phosphate compounds and titanium dioxide in a PEEK matrix makes it possible to obtain a thickness of the surface layer of approximately 1 μm. The same treatment applied to an implant with an identical shape but made of material additionally comprising 10% poly(amide-imide) fibers or calcium silicate fibers makes it possible to obtain an active surface layer thickness of 3.6 μm.

The description above illustrates clearly that by its different characteristics and their advantages, this invention achieves its objectives. In particular, it makes it possible to obtain an injection molded and reinforced implant comprising a surface osseointegration layer with thickness that is at least three times the thickness that can be achieved without reinforcement.

1-17. (canceled)

18. A part adapted for in vivo endosseous implantation comprising a material comprising:
a thermoplastic organic binder, and
a fiber charge;
wherein fibers located in a surface layer of said part are mostly delaminated from the binder over all or part of their length.

19. The part according to claim 18, wherein the fiber charge comprises nanofibers or nanotubes.

20. The part according to claim 18, wherein the fiber charge comprises microfibers.

21. The part according to claim 18, wherein the binder comprises polyetheretherketone.

22. The part according to claim 18, wherein the fibers are made of a polymer of a family of aromatic polyamides.

23. The part according to claim 22, wherein the fibers are made of poly(amide-imide).

24. The part according to claim 18, comprising fibers made of calcium silicate (Ca₂SiO₄).

25. The part according to claim 18, wherein the thickness of the surface layer is greater than or equal to 2000 nanometers.

26. The part according to claim 18, wherein the material further comprises a charge of components made from calcium and phosphate.

27. The part according to claim 26, wherein the charge of calcium-based components is made up of tricalcium phosphate Ca₃(PO₄)₂ with a hexagonal 0 structure.

28. The part according to claim 26, wherein the material further comprises a zeolite charge.

29. A method for manufacturing the part according to claim 18, comprising the steps of:
mixing a thermoplastic polymer and a fiber charge by extrusion and granulation to provide a granulate;
molding the part by injection in a mold comprising a cavity with a shape configured for the granulate to provide a blank; and
submitting the blank to ultrasonic pickling baths to delaminate the fibers in the surface layer.

30. A method for making a granulate suitable for manufacturing the part according to claim 29, comprising the steps of:
mixing by extrusion and granulation of a thermoplastic polymer and a charge comprising calcium-based components to obtain a first granulate; and
mixing the first granulate by extrusion and granulation with the fiber charge to obtain a final granulate suitable for injection.

31. The method according to claims 29, wherein the fiber charge ranges between 5% and 15% by mass of a mixture of thermoplastic polymer and the fiber charge.

32. The method according to claims 30 wherein the fiber charge ranges between 5% and 15% by mass a mixture of thermoplastic polymer and the fiber charge.

33. A granulate or compound for manufacturing a part according to claim 26 by plastic injection molding, comprising:
a polyetheretherketone (PEEK) polymer binder;
a 10% to 20% charge by mass of compounds containing calcium and zeolites; and
a 5% to 15% fiber charge.

34. The method according to claim 29, wherein the step of submitting further comprises the steps of, in the stated order:
immersing the blank in a bath subjected to ultrasound to reduce particles containing iron; and
immersing the blank in a solvent of the binder subjected to ultrasound.

35. The method according to claim 29, wherein the step of submitting further comprises the steps of, in the stated order:
immersing the blank in the following baths subjected to ultrasound:
Hydrochloric acid;
Acetone;
Hydrogen peroxide; and
rinsing in a bath of water subject to ultrasound between the immersions.
